(19) United States
(12) Patent Application Publication
(10) Pub. No.: US 2026/0014196 A1
(43) Pub. Date: Jan. 15, 2026

(54) COMPOSITIONS OF MOLECULAR HYDROGEN, OXYHYDROGEN AND THERAPEUTIC USES THEREOF

(71) Applicant: Elemental Biologic LLC, Scottsdale, AZ (US)

(72) Inventors: Kris Kesler, Phoenix, AZ (US); Lindsay Kesler, Phoenix, AZ (US); Thomas J. Johnson, Phoenix, AZ (US); Douglas Harkey, Phoenix, AZ (US)

(73) Assignee: Elemental Biologic LLC, Scottsdale, AZ (US)

(21) Appl. No.: 19/330,757

(22) Filed: Sep. 16, 2025

Related U.S. Application Data
(63) Continuation-in-part of application No. 19/275,968, filed on Jul. 21, 2025.
Application No. 19/275,968 is a continuation of application No. 17/156,317, filed on Jan. 22, 2021, now abandoned.
(60) Provisional application No. 62/965,504, filed on Jan. 24, 2020.

(51) Int. Cl. A61K 33/00; A61F 9/00; A61K 9/00; A61P 27/04

(52) U.S. Cl. CPC A61K 33/00; A61F 9/0008; A61K 9/0048; A61P 27/04

(57) ABSTRACT

Provided herein are compositions and methods that include hydrogen gas or a mixture of hydrogen gas and oxygen gas or a solution of hydrogen and oxygen for ocular therapies and tissue healing. The compositions can be incorporated into a container with a quick-release seal allowing for efficient topical administration of the medicaments of the composition to tissues desired for treatment, such as the human eye.

### RELATED APPLICATIONS

The present application is a Continuation-in-Part of U.S. patent application Ser. No. 19/275,968, which is a Continuation-in-Part of U.S. patent application Ser. No. 17/156,317 filed Jan. 22, 2021 which claims priority to U.S. provisional patent application No. 62/965,504 filed Jan. 24, 2020, the contents of which are incorporated herein by reference.

### TECHNICAL FIELD

The present disclosure generally relates to ocular therapeutics, and more specifically, to formulations and methods that use hydrogen (H²) to treat ocular conditions.

### BACKGROUND

The human eye is an organ of the sensory nervous system that reacts to visible light and allows the use of visual information for various purposes including seeing things, keeping balance, and maintaining circadian rhythm. The human eye is prone to a variety of diseases, disorders, syndromes and other undesirable conditions. Eye diseases are conditions that affect any part of your eye, and include conditions that affect the structures immediately around your eyes. These conditions can be acute (i.e., they develop quickly) or chronic (i.e., they develop more slowly and last a long time). Common eye conditions include, for example, cataracts, refractive errors, glaucoma, age-related degenerations and retinopathies.

In general, eye diseases and vision disorders are extremely common. The World Health Organization estimates that over 2.2 billion people have some form of vision impairment or blindness. One reason that eye diseases are so common is that eyes do not exist in isolation from the rest of the body. Many conditions that affect one's eyes are the result of or in connection with conditions affecting other body systems. That's why there are hundreds of different conditions that can affect the eyes. Some of these conditions can cause inflammation or other potentially harmful and negative effects that can be deleterious to the eye and result in long-term issues that can affect an individual's vision and comfort. Thus, there is a need for treatments for individuals who suffer from an eye disease, disorder, syndrome or other undesirable condition.

Current methods for treating and mitigating eye conditions are generally unsatisfactory in a number of respects and can result in a patient suffering one or more adverse effects. These adverse effects can include issues with toxicity to the eye or surrounding tissues. Patients can also suffer from allergic reactions following administration of the composition used to treat the eye condition. For example, steroids, a current drug class used to reduce inflammation, are widely known to cause such side-effects as cataracts, infections and elevated intraocular pressure.

Additionally, many medications available for use to treat diseases and syndromes of the eye do not work in all patients. For example, some dry eye prescription medications are considered to be largely clinically ineffective in reducing patient symptoms and clinical signs of the disease. Furthermore, the use of eye-drops and other conventional delivery methods are difficult to use by the patient and often result in incomplete contact of the treatment solution with the eye being treated. Thus, there is a long-felt need for a treatment for diseases and syndromes of the eye, including ones applied topically that are more therapeutically effective, better tolerated, and safer than the traditional treatments currently available.

Molecular hydrogen (H₂) is a flammable, colorless, odorless gas that can act as a reducing agent under certain circumstances. It was previously considered physiologically inert in mammalian cells and was not thought to react with active substrates in biological systems. Recently, H₂ has emerged as a novel medical gas with potentially broad applications. H₂ can modulate signal transduction across multiple pathways. It can favorably reduce healing and reduces inflammation. More specifically it down regulates inflammatory cytokines, reduces apoptosis, and is a strong antioxidant reducing the level of toxic hydroxyl radicals. However, the molecular mechanisms and molecular targets remain unclear.

The present invention includes formulations and methods that use molecular hydrogen for treating various ailments, including ocular diseases. In aspects, the formulations and methods also include molecular oxygen. Accordingly, the methods described herein overcome the limitations of conventional ocular treatments.

### SUMMARY

The inventions described and claimed herein have many attributes and embodiments including those set forth or described or referenced in this brief summary. The inventions described and claimed herein are not limited to, or by, the features or embodiments identified in this summary, which is included for purposes of illustration only and not restriction.

Embodiments include the use of molecule hydrogen (H²) on the eye and body to reduce inflammation, mitigate corneal micro-cysts, minimize scarring, decrease apoptosis, promote re-epithelialization, and reduce vascular endothelial growth factor on the surface and intra ocularly. Embodiments also include compositions, for example gels, ointments, liquid-based or gaseous compositions, emulsions, vapors, liquids, frozen solutions, and delivery systems adapted to treat ocular conditions.

Various embodiments of the present disclosure relate to therapeutically effective compositions and delivery methods for treating ocular conditions utilizing a solution of oxyhydrogen (2:1 HHO ((H₂)₂:O₂)) or molecular hydrogen (H₂), a saline solution (e.g., a phosphate-buffered saline or ringer's solution) or distilled H₂O, and an optional third or more component's (such as mineral oil, aloe, propylene glycol, polyethylene glycol, or ophthalmic lubricants). In some embodiments, the compositions are provided as an emulsion. In various embodiments, the compositions provided herein comprise 0.5-10.0 ppm hydrogen (H₂). In some embodiments, the compositions comprise 0.5-1.6 ppm hydrogen gas. In other embodiments, the compositions are delivered using a single-use cup, container or goggles configured to tightly fit over a human eye region and are configured with a quick-release seal that allows the HHO or molecular hydrogen (H₂) included in the composition to immediately bathe and medicate a patient's eye. In aspect, molecule hydrogen (H²) is added to medications for topical, intracameral, intravitreal, or systemic administration.

In addition to aqueous and gas forms, another mode of application that may prove effective is H₂ or HHO frozen in a solution. A super saturated solution of water and molecular hydrogen gradually diffuses H₂ over hours until it becomes “flat” like an open soda losing CO₂, Super saturated H₂ solution can be frozen and then melted and still retain a therapeutic level of H₂. Ice containing H₂ could be applied to the eye. This mode of application could have a dual acting anti-inflammatory mechanism (thermal and H₂). In addition, H₂ suspended in a frozen solution may be an effective way of storing and transporting this super saturated solution. (this may be an especially effective mode of application for oral ailments-think super saturated H₂ popsicles).

Accordingly, aspects include a composition of (a) molecular hydrogen (H₂); and (b) saline or sterile water. The composition can also include oxygen gas (O₂). The molecular hydrogen can be present in an amount of 0.5 to 10.0 ppm. Alternatively, the molecular hydrogen can be present in an amount of 5 to 10 ppm. The molecular hydrogen can present in lower amounts (e.g., 0.5 to 0.6 ppm).

The composition can also include mineral oil, aloe, polyethylene glycol and/or propylene glycol. In aspects, the pH is about 5.5, about 6.5, about 7, about 7.5 or about 8. In aspects, the pH is at least 6.5. Alternatively, the pH is at least 7.2-7.4. In aspects, the oxygen gas is at a concentration of at least 9-13 mg/L.

Embodiments also include an ocular delivery system. The system can include a container with an opening configured to substantially fit over an eye of a subject, a quick-release seal (removably attached to and providing a water-proof seal for the opening of the container) and a composition disposed within the container. The composition can include (a) 0.5-10.0 ppm molecular hydrogen (H₂) and (b) saline or sterile water. In aspects, the composition is another solution as described herein. H₂ concentration in solution may be worth mentioning;

In aspects, molecular hydrogen (H₂) is present in the formulations described herein at about 1.6 ppm. In aspects, molecular hydrogen (H₂) is present at 0.3 ppm, 0.4 ppm. 0.5 ppm, 0.6 ppm, 0.8 ppm. 1.0 ppm, 1.2 ppm, 1.4 ppm. 1.6 ppm or higher. In aspects, H₂ is added to the formulations described herein at high pressure.

Embodiments also include a method of healing or promoting healing of an ocular surface. The method can include steps of (a) providing an ocular delivery system as substantially described herein, (b) placing the ocular delivery system over the eye of a subject and (c) deploying the quick-release seal to expose the eye of the subject to the composition.

Embodiments also include an ocular delivery that includes goggles or eye cup configured to be worn airtight over a subject's or patient's orbital bones and cover a subject's eyes. The goggles can be configured to deliver gaseous or aqueous molecular hydrogen to a subject's eyes.

In some embodiments, the compositions described herein can be administered as a drop (i.e., via topical instillation), an ointment, an eye soak, an emulsion, a gas, a vapor, a mist, an aerosol, a gel, a bottled or canned solution, a bag on valve system or other art-recognized equivalents. In aspects, H₂ is combined with another medication. In aspects, the compositions described herein are combined with one or more antibiotics, one or more steroids, or a combination thereof. H₂ can also be beneficial when combined with lubricants, and medications both over the counter and prescription.

In aspects, the hydrogen in the solution down-regulates pro-inflammatory cytokines associated with various ocular conditions. In aspects, the compositions described herein are administered with one or more additional medicaments such as a VEGF inhibitor.

In other embodiments, the compositions are administered via injection (e.g., intravitreal, conjunctival or retrobulbar injection). The compositions of the injectable may be combined with one or more carriers or diluents, antibiotics, steroids, other medications or a combination thereof. A device creating or diffusing H₂ can be injected into the body or the eye. The injection device can be any art-recognized injection device, including a needled syringe, a needleless syringe, a jet injector, a pen injector, a microneedle device, a nanoneedle device, a catheter, a cannula, a subtenon injector or the like.

In embodiments, the present disclosure can be used for the reduction of VEGF (vascular endothelial growth factor) as well as the treatment of macular degeneration and diabetic retinopathy.

In further embodiments, the present disclosure can be used for treatment of, or concurrent with treatment of, ocular sequela, iritis, bacterial infection, viral infection, scleritis, epi-scleritis, allergic eye diseases, dry eye/meibomian gland dysfunction, dry-eye diseases including evaporative, mucin and/or aqueous deficient as well as filamentary keratitis, chemical burns, thermal burns of the eye, uveitis, corneal micro cysts, corneal edema, cystoid macular edema, vitreous floaters, giant papillary conjunctivitis, medicamentosa, central serous chorioretinopathy, post-surgical inflammation, post-operative inflammation, reaction to antibiotic/antiviral treatment, contact-lens-related irritation, red eye, sequela, inflammation (of any origin), superior limbic keratoconjunctivitis (SLK) and as a VEGF inhibitor.

In further embodiments, the present disclosure can be used for treatment of, or concurrent with treatment of, oral lesions, receding gums, cavities (tooth decay), gum (periodontal) disease, cold sores, denture stomatitis, dry mouth, geographic tongue, fungal/yeast growth, human papilloma virus (HPV), ulcers and oral cancer. It could also be used to speed healing after dental procedures or oral surgery. In aspects, the formulations and methods described herein can be used to reduce inflammation.

In some embodiments, the present disclosure may be used to promote ocular healing needed as a result of physical damage from another ocular treatment, or irritation due to an ocular disease from another ocular treatment.

A water-based solution can be super saturated with H₂ and then bottled or canned to use by the patient (like a can of soda). Likewise, an ointment could be saturated with H₂ and packaged in a container that holds H₂. In aspects, a tablet composed of ceramic and/or magnesium or other metal is placed in a solution to produce a super saturated H₂ solution. A home electrolysis device can also be sold to the consumer to make H₂ solution. In aspects, a device is implanted into the body to produce H₂.

The invention relates to a kit or package comprising the compound of the present invention or the pharmaceutical composition of the present invention.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The present disclosure will hereinafter be described in conjunction with the appended figures, wherein like numerals denote like elements, and:

FIG. 1 is schematic overview of an ocular delivery system in accordance with various embodiments.

FIG. 2A shows, in sequence with FIG. 2B, the application of an HHO or molecular hydrogen (H₂) solution using an ocular delivery system in accordance with various embodiments.

FIG. 2B shows removal of the quick-release seal attached to the opening of the container in FIG. 2A.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but no other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of” when used in a claim of this invention is not intended to be interpreted to be equivalent to “comprising.” Unless the context indicates otherwise, it is specifically intended that the various features of the invention described herein can be used in any combination.

Consequently, alternative language and synonyms may be used for any one or more of the terms discussed herein. Nor is any special significance to be placed upon whether or not a term is elaborated on or discussed herein. Synonyms for certain terms are provided. A recital of one or more synonyms does not exclude the use of other synonyms. The use of examples anywhere in this specification, including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “at least” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “medicament,” “active agent” or “active ingredient” refers to a substance, compound, or molecule, which is biologically active or otherwise, induces a biological or physiological effect on a subject to which it is administered. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “subject” or “patient” refers to any single animal, more preferably a mammal (including such non-human animals as, for example, dogs, cats, horses, rabbits, zoo animals, cows, pigs, sheep, and non-human primates) for which treatment is desired.

In embodiments, “an effective amount” refers, without limitation, to the amount of the defined component sufficient to achieve the desired therapeutic result. In an embodiment, that result can be effective ocular treatment.

In an embodiment, as used herein, the terms “treating,” “treatment” and the like are used herein, without limitation, to mean obtaining a desired pharmacologic and/or physiologic effect. The effect may be prophylactic in terms of completely or partially preventing a disorder, sign, or symptom thereof, and/or may be therapeutic in terms of amelioration of the symptoms of the disease or infection, or a partial or complete cure for a disorder and/or adverse effect attributable to the disorder.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The term “treatment” refers to the application of one or more specific procedures used for the attempted amelioration of a disease. In certain embodiments, the specific procedure is the administration of one or more therapeutic agents. “Treatment” of a subject (e.g., a mammal, such as a human) or a cell is any type of intervention used in an attempt to alter the natural course of the individual or cell. Treatment includes, but is not limited to, administration of a composition (e.g., a pharmaceutical composition), and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. An “effective amount” or “therapeutically effective amount” refers to an amount of therapeutic compound, such as hydrogen gas or hydrogen gas and oxygen gas, administered to a subject, either as a single dose or as part of a series of doses, which is effective to produce a desired therapeutic effect.

The term “ailment” or “syndrome” refers to a disease, disorder, illness or medical condition. An ailment can be, for example, an ophthalmic or ocular ailment such as cystoid macular edema, retinal dystrophy, dry eye, red eye, macular degeneration.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

Iritis is generally considered an inflammation of the iris: a contractile membrane perforated by the pupil, which adjusts to control the amount of light reaching the retina and forms the colored portion of the eye.

Scleritis is generally considered inflammation of the sclera of the eye, where the sclera is generally defined as the “white” of the eye, a tough outer coat of the eye that covers the eyeball except for the cornea.

Episcleritis is generally referred to as an inflammatory disease affecting the episclera, which is the outermost layer of the sclera, composed of loose, fibrous, elastic tissue.

Dry eye is generally defined as an eye disease caused by decreased tear production and inflammation.

Uveitis is generally defined as inflammation of the uvea, which is the middle of the three concentric layers that make up the eye.

Red eye is commonly referred to as redness of the eye, most typically the sclera or conjunctiva. Red eye is typically caused by trauma or injury to the eye, dryness of the eyes, over-wearing or improper wearing of contact lenses, eye strain, computer eye strain, smoking, lack of sleep, or swimming in chlorinated pools or bodies of water containing irritants or pathogens.

Corneal micro cysts are generally referred to as small pouches or sacs containing fluid or other matter on or within the cornea. They occur in eyes and are a secondary result from, in an embodiment, antibody drug conjugate use.

The macula is generally defined as an oval yellow spot near the center of the retina of the human eye and can contain two or more layers of ganglion cells, responsible for detailed central vision.

Giant papillary conjunctivitis is generally defined when the inside of the eyelid becomes red, swollen and irritated is caused by contact lens use.

Medicamentosa is generally defined as eye irritation due to medications applied to the eye. Medicamentosa may also be caused by preservatives or carrier agents in the eye medication which further irritate the eye.

The term “oxyhydrogen” or “HHO” refers to a mixture of hydrogen (H₂) and oxygen (O₂) gases. In this regard, the phrase “oxyhydrogen” as used herein refers to any gaseous combination of diatomic hydrogen and oxygen (H₂, O₂), and the phrase “oxyhydrogen solution” refers to any combination of those gases with a liquid constituent, regardless of whether the resulting mixture is properly classified as a suspension, a solution, a colloid, or any other such mixture of gaseous and liquid components. Further, the term “knallgass” or “knallgas” (also referred to as “Oxyhydrogen” or “HHO”) is used in the sense of an oxyhydrogen gas that has a H H O (H₂O₂ is hydrogen peroxide and is harmful to the eye) ratio substantially equal to 2:1, which may be produced, for example, through dissociation of water molecules via water electrolysis: H₂O→H+H+O.

HHO is, by definition, the gaseous mixture of hydrogen and hydrogen and oxygen as the result of the disassociation of the covalent bonds of a water molecule through electrolysis or resonant frequency with high voltage and low current. Thus, in some embodiments, provided herein are compositions that include a hydrogen gas and a vehicle. The compositions can be sterile.

The term “compound” refers to a combined mixture of multiple ingredients. A compound may further refer to a customized formulation of ingredients designed for therapeutic, prophylactic, preventative and aseptic purposes.

The term “oromucosal use” or “oromucosal administration” refers to the application of a drug or pharmaceutical agent through the oral mucosa, specifically the oral cavity and/or the pharynx, including one or more of the nasopharynx, oropharynx or laryngopharynx.

The term “intranasal use” or “intranasal administration” refers to the application of a drug or pharmaceutical agent to the nose, nasal cavity and/or nasopharynx. The esophagus may also be targeted using embodiments of the present invention.

The term “intrarectal use” or “intrarectal administration” or “rectal use” or “rectal administration” refers to the application of a drug or pharmaceutical agent to the rectal cavity. The term “vaginal use” or “vaginal administration” refers to the application of a drug or pharmaceutical agent to the vaginal cavity.

All numerical designations, e.g., pH, temperature, time, concentration, and molecular weight, including ranges, are to be understood as approximations in accordance with common practice in the art. When used herein, the term “at least” may connote variation (+) or ( ) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13th Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

### DETAILED DESCRIPTION

The following detailed description of the present disclosure is merely exemplary in nature and is not intended to limit the present disclosure or the application and uses of the present disclosure. Furthermore, there is no intention to be bound by any theory presented in the preceding background or the following detailed description.

Chronic/persistent oxidative stress can be associated with the pathogenesis of many lifestyle-related diseases, aging and cancer. However, many clinically tested antioxidants exhibit high toxicity levels that limit their use. Recent studies have presented evidence that molecular hydrogen (H₂) has selective antioxidant properties and can have beneficial effects in treating many ailments. It has been discovered that many eye conditions are ameliorated, without being bound by theory, by anti-inflammatory therapeutics and applications of hydrogen gas or a mixture of hydrogen gas and oxygen gas. Accordingly, embodiments include compositions and methods of treating eye conditions using hydrogen gas or a mixture of hydrogen gas and oxygen gas.

The compositions and methods described herein can utilize H₂ to reduce and/or downregulate pro-inflammatory cytokines and reduce apoptosis associated with various ocular conditions, such as allergies, dry-eye, epi-scleritis, scleritis, uveitis, postsurgical inflammation, antibiotic treatments, and contact-lens-related irritation (e.g., red-eye). Treatable conditions include, for example, corneal micro-cysts, including those caused by the use of an antibody drug conjugate, iritis, bacterial infection, viral infection, scleritis, episcleritis, burns (both chemical, thermal and ultraviolet) allergies, dry-eye, uveitis, post-surgical or post-operative inflammation or healing, reaction to antibiotic/antiviral treatment, and contact-lens-related irritation or issues, red eye, and post-injury recovery, post-trauma recovery or a combination thereof. The compositions provided herein can be used to reduce vascular endothelial growth factor (VEGF), and the treatment of macular degeneration, age-related macular degeneration (AMD), dry AMD, wet AMD, diabetic retinopathy, or other ocular conditions or diseases, or a combination thereof.

Accordingly, embodiments of the invention include formulations and methods that use of H₂ to promote health and/or treat an ailment. In aspects, the formulations described herein reduce inflammation, reduce apoptosis, minimize scarring, promote re-epithelialization, and reduce vascular endothelial growth factor on the surface and intra ocularly.

Embodiments also include compositions and delivery methods for treating ocular conditions utilizing a solution of oxyhydrogen (2:1 HHO ((H₂)₂:O₂)) or molecular hydrogen (H₂), a saline solution (e.g., a phosphate-buffered saline or ringer's solution) or distilled H₂O, and an optional third or more component's (e.g., mineral oil, aloe, propylene glycol, polyethylene glycol, or ophthalmic lubricants).

Aspects further include methods of treating various ailments using compositions that include oxyhydrogen (2:1 HHO ((H₂)₂:O₂)), molecular hydrogen (H₂), a saline solution (e.g., a phosphate-buffered saline) and/or purified/distilled H₂O. The compositions can optionally include additional components (e.g., a mineral oil, aloe, propylene glycol, polyethylene glycol, or ophthalmic lubricants). In aspects, the compositions include 0.5-10.0 ppm hydrogen (H₂).

In a further aspect, the composition comprises and/or includes 0.5 ppm hydrogen (H₂), 1 ppm hydrogen (H₂), 1.5 ppm hydrogen (H₂), 2 ppm hydrogen (H₂), 2.5 ppm hydrogen (H₂), 3 ppm hydrogen (H₂), 3.5 ppm hydrogen (H₂), 4 ppm hydrogen (H₂), 4.5 ppm hydrogen (H₂), 5 ppm hydrogen (H₂), 5.5 ppm hydrogen (H₂), 6 ppm hydrogen (H₂), 6.5 ppm hydrogen (H₂), 7 ppm hydrogen (H₂), 7.5 ppm hydrogen (H₂), 8 ppm hydrogen (H₂), 8.5 ppm hydrogen (H₂), 9 ppm hydrogen (H₂), 9.5 ppm hydrogen (H₂) or 10 ppm hydrogen (H₂).

In a another aspect, the composition comprises and/or includes at least 0.5 ppm hydrogen (H₂), at least 1 ppm hydrogen (H₂), at least 1.5 ppm hydrogen (H₂), at least 2 ppm hydrogen (H₂), at least 2.5 ppm hydrogen (H₂), at least 3 ppm hydrogen (H₂), at least 3.5 ppm hydrogen (H₂), at least 4 ppm hydrogen (H₂), at least 4.5 ppm hydrogen (H₂), at least 5 ppm hydrogen (H₂), at least 5.5 ppm hydrogen (H₂), at least 6 ppm hydrogen (H₂), at least 6.5 ppm hydrogen (H₂), at least 7 ppm hydrogen (H₂), at least 7.5 ppm hydrogen (H₂), at least 8 ppm hydrogen (H₂), at least 8.5 ppm hydrogen (H₂), at least 9 ppm hydrogen (H₂), at least 9.5 ppm hydrogen (H₂) or at least 10 ppm hydrogen (H₂).

In a another aspect, the composition comprises and/or includes at least 0.5 ppm hydrogen (H₂), at least 1 ppm hydrogen (H₂), at least 1.5 ppm hydrogen (H₂), at least 2 ppm hydrogen (H₂), at least 2.5 ppm hydrogen (H₂), at least 3 ppm hydrogen (H₂), at least 3.5 ppm hydrogen (H₂), at least 4 ppm hydrogen (H₂), at least 4.5 ppm hydrogen (H₂), at least 5 ppm hydrogen (H₂), at least 5.5 ppm hydrogen (H₂), at least 6 ppm hydrogen (H₂), at least 6.5 ppm hydrogen (H₂), at least 7 ppm hydrogen (H₂), at least 7.5 ppm hydrogen (H₂), at least 8 ppm hydrogen (H₂), at least 8.5 ppm hydrogen (H₂), at least 9 ppm hydrogen (H₂), at least 9.5 ppm hydrogen (H₂) or at least 10 ppm hydrogen (H₂).

In a another aspect, the composition comprises and/or includes no more than 0.5 ppm hydrogen (H₂), no more than 1 ppm hydrogen (H₂), no more than 1.5 ppm hydrogen (H₂), no more than 2 ppm hydrogen (H₂), no more than 2.5 ppm hydrogen (H₂), no more than 3 ppm hydrogen (H₂), no more than 3.5 ppm hydrogen (H₂), no more than 4 ppm hydrogen (H₂), no more than 4.5 ppm hydrogen (H₂), no more than 5 ppm hydrogen (H₂), no more than 5.5 ppm hydrogen (H₂), no more than 6 ppm hydrogen (H₂), no more than 6.5 ppm hydrogen (H₂), no more than 7 ppm hydrogen (H₂), no more than 7.5 ppm hydrogen (H₂), no more than 8 ppm hydrogen (H₂), no more than 8.5 ppm hydrogen (H₂), no more than 9 ppm hydrogen (H₂), no more than 9.5 ppm hydrogen (H₂) or no more than 10 ppm hydrogen (H₂).

In aspects, the compositions comprise and/or include 0.5-1.6 ppm hydrogen gas.

### Delivery Systems

The compositions can be administered using a single-use cup, container or goggles configured to tightly fit over a human eye region to allow the HHO or molecular hydrogen (H₂) in the composition to cover a surface area medicate of an eye. It can also be administered via an eye wash.

Accordingly, the present invention provides devices, systems and methods for treating ocular and ophthalmic diseases, syndromes and disorders through application of therapeutic compositions comprising hydrogen, oxygen and hydrogen, or a combination of either or both of the prior with a buffer, diluent, carrier agent, steroid, solution, therapeutic, medicament, lubricant, solvent or antibiotic.

A pharmaceutical composition disclosed herein may comprise molecular hydrogen (H2) and a solvent in an amount sufficient to dissolve molecular hydrogen (H2) disclosed herein. In other aspects of this embodiment, a pharmaceutical composition disclosed herein may comprise molecular hydrogen (H2) dissolved in a solvent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a pharmaceutical composition disclosed herein may comprise molecular hydrogen (H2) dissolved in a solvent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

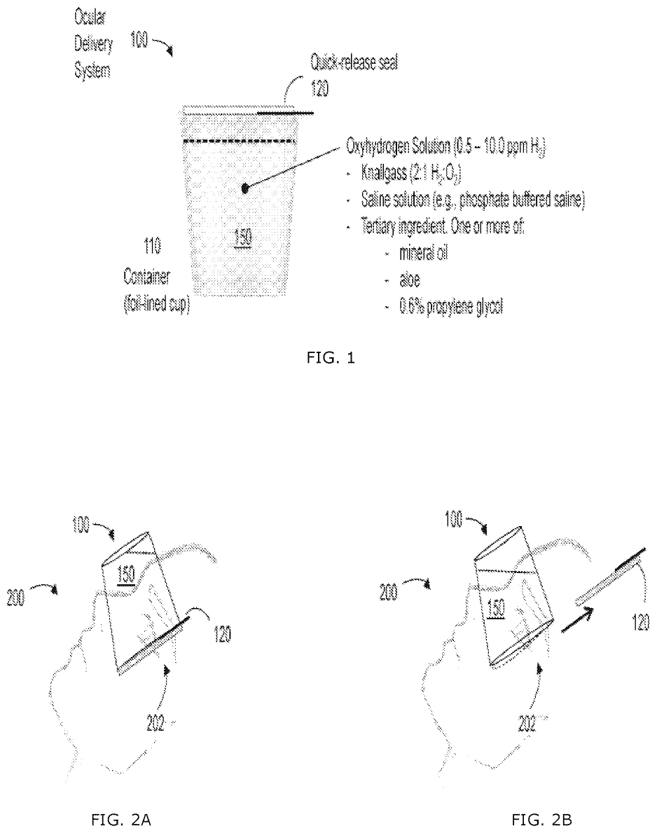

Embodiments also include delivery systems and methods for administering compositions to treat ocular conditions. Referring first to FIG. 1, an ocular delivery system 100 in accordance with various embodiments generally includes a container 110 (e.g., a foil-lined, one-time-use sterile container) having a quick-release seal 120 covering its top opening. In aspects, the geometry of the top opening (e.g., size and shape) substantially fits within the orbit over a human eye in a reasonably watertight manner. While FIG. 1 illustrates a traditional cup-shaped container 110, the present disclosure is not so limited. Container 110 can be, for example, elliptical, curvilinear, pyramidal, ovular, concave, convex or any other shape or orientation suitable for fitting over a human eye.

The quick-release seal 120 can include any combination of components that allow a patient or caregiver to quickly and easily release the eye-treatment solution when the container 110 is in contact with the patient's face or eye so that the solution can effectively flow over and bathe and medicate the patient's eye. Various adhesives and seal materials can be used such as rubber, elastomeric rubber, polymers, copolymers, synthetic polymers, synthetic copolymers, silicone, neoprene, nitriles, fluorocarbons, plastics, and any other art-recognized equivalent suitable for forming a seal or gasket.

Disposed within container 110 is an eye-treatment solution (e.g., an HHO or molecular hydrogen (H₂) solution) 150. The solution can include, for example, oxyhydrogen or H₂, a saline solution, and one or more optional components such as mineral oil, aloe, propylene glycol, polyethylene glycol, castor oil, or other acceptable ophthalmic lubricants.

In some embodiments, the hydrogen gas is a combination of hydrogen gas and oxygen gas (O₂). When hydrogen and oxygen gases are both present, the ratio of the gases may be at least 2:1 H₂:O₂. In an embodiment, the ration of the gases may be at least 0.1:1 H₂:O₂, at least 0.2:1 H₂:O₂, at least 0.3:1 H₂:O₂, at least 0.4:1 H₂:O₂, at least 0.5:1 H₂:O₂, at least 0.6:1 H₂:O₂, at least 0.7:1 H₂:O₂, at least 0.8:1 H₂:O₂, at least 0.9:1 H₂:O₂, at least 1:1 H₂:O₂, at least 1.1:1 H₂:O₂, at least 1.2:1 H₂:O₂, at least 1.3:1 H₂:O₂, at least 1.4:1 H₂:O₂, at least 1.5:1 H₂:O₂, at least 1.6:1 H₂:O₂, at least 1.7:1 H₂:O₂, at least 1.8:1 H₂:O₂, at least 1.9:1 H₂:O₂, at least 2:1 H₂:O₂, at least 2.1:1 H₂:O₂, at least 2.2:1 H₂:O₂, at least 2.3:1 H₂:O₂, at least 2.4:1 H₂:O₂, at least 2.5:1 H₂:O₂, at least 2.75:1 H₂:O₂, at least 3:1 H₂:O₂ or greater.

In another embodiment, the ration of the gases may be about 0.1:1 H₂:O₂, about 0.2:1 H₂:O₂, about 0.3:1 H₂:O₂, about 0.4:1 H₂:O₂, about 0.5:1 H₂:O₂, about 0.6:1 H₂:O₂, about 0.7:1 H₂:O₂, about 0.8:1 H₂:O₂, about 0.9:1 H₂:O₂, about 1:1 H₂:O₂, about 1.1:1 H₂:O₂, about 1.2:1 H₂:O₂, about 1.3:1 H₂:O₂, about 1.4:1 H₂:O₂, about 1.5:1 H₂:O₂, about 1.6:1 H₂:O₂, about 1.7:1 H₂:O₂, about 1.8:1 H₂:O₂, about 1.9:1 H₂:O₂, about 2:1 H₂:O₂, about 2.1:1 H₂:O₂, about 2.2:1 H₂:O₂, about 2.3:1 H₂:O₂, about 2.4:1 H₂:O₂, about 2.5:1 H₂:O₂, about 2.75:1 H₂:O₂, about 3:1 H₂:O₂ or greater.

In further embodiments, the ration of the gases may be no more than 0.1:1 H₂:O₂, no more than 0.2:1 H₂:O₂, no more than 0.3:1 H₂:O₂, no more than 0.4:1 H₂:O₂, no more than 0.5:1 H₂:O₂, no more than 0.6:1 H₂:O₂, no more than 0.7:1 H₂:O₂, about 0.8:1 H₂:O₂, about 0.9:1 H₂:O₂, about 1:1 H₂:O₂, about 1.1:1 H₂:O₂, about 1.2:1 H₂:O₂, about 1.3:1 H₂:O₂, about 1.4:1 H₂:O₂, about 1.5:1 H₂:O₂, about 1.6:1 H₂:O₂, about 1.7:1 H₂:O₂, about 1.8:1 H₂:O₂, about 1.9:1 H₂:O₂, about 2:1 H₂:O₂, about 2.1:1 H₂:O₂, about 2.2:1 H₂:O₂, about 2.3:1 H₂:O₂, about 2.4:1 H₂:O₂, about 2.5:1 H₂:O₂, about 2.75:1 H₂:O₂, about 3:1 H₂:O₂ or greater.

In various embodiments, the hydrogen gas is present at an amount of at least 0.5-10 ppm. In some embodiments, the hydrogen gas is present at an amount of at least 0.5-2.0 ppm. In other embodiments, the hydrogen gas is present at an amount of at least 0.7-1.6 ppm. In further embodiments, the hydrogen gas is present, but in an amount not more than 2.0 ppm, i.e., less than 2.0 ppm. In some embodiments, the hydrogen gas is present at an amount of at least 1.6 ppm. In further embodiments, the hydrogen gas is present at an amount between 4.5 to 6.5 ppm. In other embodiments, the hydrogen gas is present at an amount between 5.0 to 6.0 ppm.

In some embodiments, the hydrogen gas is present at an amount of at least 2 ppm, at least 3 ppm, at least 4 ppm, at least 5 ppm, at least 5-10 ppm, or at least 10 ppm. In further embodiments, the hydrogen gas is present at an amount of at least 20 ppm.

In some embodiments, the vehicle is water. The water can be distilled water, deionized water, or both. In some embodiments, the vehicle is a phosphate buffered saline, which may include calcium, magnesium, or both. In various embodiments, the vehicle is a ringer's solution, which may include magnesium. In other embodiments, the ringer's solution is a lactated ringer's solution. In some embodiments, the vehicle may be sterile.

In some embodiments, the pH of the compositions provided herein may be at least 6.0-8.0. In further embodiments, the pH of the compositions is at least 6.5-7.5. In some embodiments, the pH of the compositions is at least 6.5. In other embodiments, the pH of the compositions is at least 7.2. In yet other embodiments, the pH of the compositions is at least 7.3-7.4. In further embodiments, the pH of the compositions is 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 102, 10.2, 10.3, 10.4, 10.5, 10.6, 10.7, 10.8, 10.9, 11, 11.1, 11.2, 11.3, 11.4, 11.5, 11.6, 11.7, 11.8, 11.9 or 12.

In some embodiments, the compositions provided herein further include one or more additional components. An additional component can be an ophthalmic lubricant. In other embodiments, the additional component is, independently, mineral oil, aloe, propylene glycol, or polyethylene glycol.

In embodiments, the composition is a mixture of at least 0.7-1.6 ppm H₂ in sterile distilled water having a pH of at least 7.2. In some embodiments, the composition is a mixture of at least 0.7-1.6 ppm H₂, O₂, and sterile distilled water, wherein the ratio of H₂:O₂ is at least 2:1, and the pH of the composition is at least 7.2. In other embodiments, these compositions further comprise an aloe, a mineral oil, or both. In some embodiments, these compositions further comprise erythromycin.

In various embodiments, the composition is a mixture of at least 0.5-10 ppm H₂ in sterile distilled water having a pH of at least 6.5-7.5. In other embodiments, the composition is a mixture of at least 0.5-10 ppm H₂, O₂, and sterile distilled water, wherein the ratio of H₂:O₂ is at least 2:1, and the pH of the composition is at least 6.5-7.5. In further embodiments, these compositions further comprise an aloe, a mineral oil, or both. In some embodiments, these compositions further comprise erythromycin.

Distilled water may have an oxygen gas concentration of 6.5-8 mg/L. Thus, in some embodiments, the composition comprises an oxygen gas concentration of at least 9-13 mg/L. In some embodiments, the composition comprises an oxygen gas concentration of at least 9 mg/L to at least 13 mg/L. In further embodiments, the ratio of H₂:O₂ is at least 2:1, and the pH of the composition is at least 6.5-7.5, and the concentration of O₂ is at least 9-13 mg/L. In other embodiments, the O₂ concentration is at least 9 mg/L. In some embodiments, the O₂ concentration is at least 10 mg/L. In some embodiments, the O₂ concentration is at least 11 mg/L. In other embodiments, the O₂ concentration is at least 12 mg/L. In some embodiments, the O₂ concentration is at least 3 mg/L.

In further embodiments, the O₂ concentration is at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, or more.

In further embodiments, the O₂ concentration is about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, or more.

In further embodiments, the O₂ concentration is no more than 1 mg/L, no more than 2 mg/L, no more than 3 mg/L, no more than 4 mg/L, no more than 5 mg/L, no more than 6 mg/L, no more than 7 mg/L, no more than 8 mg/L, no more than 9 mg/L, no more than 10 mg/L, no more than 11 mg/L, no more than 12 mg/L, no more than 13 mg/L, no more than 14 mg/L, no more than 15 mg/L, no more than 16 mg/L, no more than 17 mg/L, no more than 18 mg/L, no more than 19 mg/L, no more than 20 mg/L, or more.

In various embodiments, eye-treatment solution comprises, overall, 0.5-10.0 ppm H₂, and the saline solution consists of medical grade phosphate-buffered saline as is known in the art. In addition to oxyhydrogen and a saline solution, eye-treatment solution 150 may also include various other ingredients (“tertiary ingredients”), such as mineral oil, aloe, and propylene glycol, each in various weight or volume-percents ranging from at least 5% to 7%, though other embodiments may incorporate other ingredients at different weight or volume percents.

In an aspect, eye-treatment solution 150 includes hydrogen or oxyhydrogen, and phosphate buffered saline combined such that the hydrogen concentration is in the range of 0.5-10.0 ppm.

In another aspect, eye-treatment solution 150 includes hydrogen or oxyhydrogen, phosphate buffered saline, and mineral oil (e.g., 1% by weight) combined such that the hydrogen concentration is in the range of 0.5-10.0 ppm.

In an embodiment, eye-treatment solution 150 includes hydrogen or oxyhydrogen, phosphate buffered saline, and an aloe (e.g., aloe vera liquid) combined such that the hydrogen concentration is in the range of 0.5-10.0 ppm.

In another embodiment, eye-treatment solution 150 includes hydrogen or oxyhydrogen, phosphate buffered saline, and 0.6% propylene glycol combined such that the hydrogen concentration is in the range of 0.5-10.0 ppm.

In yet another embodiment, eye-treatment solution 150 includes hydrogen or oxyhydrogen, phosphate buffered saline, aloe, and 0.6% propylene glycol combined such that the hydrogen concentration is in the range of 0.5-10.0 ppm.

Table 1 provides examples of selected compositions provided herein.

TABLE 1
Compositions

| | Gas (H₂ 0.7-1.6 | | |
|---|---|---|---|
| | ppm; H₂:O₂ of | | |
| | about 2:1 when | | Additional |
| Composition | present) | Vehicle | components |
| 1 | H₂ | Sterile distilled | |
| | | water; pH about | |
| | | 7.2 | |
| 2 | H₂ | Phosphate buffered | |
| | | saline (PBS) (137 | |
| | | mM NaCl, 10 mM | |
| | | phosphate, 2.7 mM | |
| | | KCl; pH 7.4) | |
| 3 | H₂ | Ringer's solution | |
| 4 | H₂ | Sterile distilled | Mineral oil |
| | | water | |
| 5 | H₂ | PBS | Mineral oil |
| 6 | H₂ | Ringer's solution | Mineral oil |
| 7 | H₂ | PBS | Mineral oil Aloe |
| | | | Propylene glycol |
| 8 | H₂ and O₂ | Sterile distilled | |
| | | water; pH | |
| | | about 7.2 | |
| 9 | H₂ and O₂ | PBS | |
| 10 | H₂ and O₂ | Ringer's solution | |
| 11 | H₂ and O₂ | Sterile distilled | Mineral oil |
| | | water | |
| 12 | H₂ and O₂ | PBS | Mineral oil |
| 13 | H₂ and O₂ | Ringer's solution | Mineral oil |
| 14 | H₂ and O₂ | PBS | Mineral oil Aloe |
| | | | Propylene glycol |

Treatment of a patient for an ocular condition generally can proceed as illustrated in FIGS. 2A and 2B. The ocular delivery system 100 is first positioned with the quick-release seal 120 facing downward and the perimeter of the opening of container 110 in contact with the patient's face 200 in and around eye area 202 (FIG. 2A). Subsequently, as shown in FIG. 2B, the patient (or caregiver) actuates quick-release seal 120 such that the eyetreatment solution 150 is allowed to freely flow, under the force of gravity, into and around the eye region 202. While some leakage of solution 150 is expected, the seal provided between the opening of container 110 and the patient's face 200 substantially restrains the solution 150 so that it can fully cover the eye region for a significant length of time, unlike eye drops that contact the eye only briefly.

Due to the improved efficacy of this delivery system, combined with the therapeutic level of hydrogen provided by eye-treatment solution 150, in an embodiment the result is an ocular treatment system the functions to downregulate pro-inflammatory cytokines associated with various ocular conditions, such as allergies, dry-eye, epi-scleritis, scleritis, uveitis, postsurgical inflammation, antibiotic treatments, and contact-lens-related irritation (e.g., red-eye).

### Administration

In a further embodiment, eye-treatment solution 150 is formulated as a mist, spray, gel, cream or aerosol. Further, the container of the present invention is configured to dispense a mist, spray, gel, cream or aerosol in the same effective manner as the other compositions disclosed and configured to operate in the same effective manner with the quick-release seal mechanism and all other components of the container as disclosed in embodiments of the present invention.

In yet other embodiments, the compositions of the present invention may be formulated as a gas or equivalent gaseous form, the gaseous composition applied to a subject's affected tissues through goggles worn at least the affected area. The affected area may be a subject or patient's eyes, and the gaseous composition is delivered inside the goggles whereby the gaseous composition saturates or envelops the subject or patient's eyes, providing effective treatment.

The compositions of the present invention can be formulated as a gel or cream, and delivered to a patient's affected area (i.e., a patient or subject's eyes) via myriad delivery means. Such delivery means include, but are not limited to, a swab, a pipette, a patch, an applicator, a dermal applicator, an ocular applicator, a needleless injector, a dispenser, a cup, a container, an open-ended vial, a squeezable container and the like. Further, the delivery means can include a quick-release seal mechanism as disclosed in embodiments of the present invention. The quick-release seal mechanism is able to release compositions of the present invention upon contact with a patient's or subject's affected area(s).

The compositions of the present invention can be formulated for injection into an injectable composition. The injectable composition of the present invention can be delivered by a needled syringe, microneedle device, nanoneedle device, cannula, catheter or other equivalent injection device. Through art-recognized modes of injection, the compositions of the present invention can be delivered under the conjunctiva or into the sub tenon's space and alternatively injected intravitreally or into a retrobulbar area (behind the eyeball) of a patient or subject. Other art-recognized areas for ocular and ophthalmic treatment are further contemplated by this disclosure for suitable injection and delivery of the compositions of the present invention to a subject or patient.

The compositions of the present invention may be formulated as a pellet that is capable of generating H₂, or H₂ and O₂. Further, the pellet of the present invention may be coated in suitable materials such as magnesium or ceramic and then injected into a subject or patient's eye.

The compositions of the present invention may be formulated as a tablet containing H₂, or H₂ and O₂, and composed of ceramic or magnesium. The tablet can be placed into a solution to produce a super-saturated H₂ solution.

The compositions of the present invention may be formulated as a super-saturated H₂ solution, or H₂ and O₂ solution, and then bottled or canned prior to use by a patient, subject or practitioner. It can also be formulated as a super-saturated H₂ solution, or H₂ and O₂ solution in a gel or cream, and then put into a tube, including a squeezable one, or other container that can be manipulated to push the gel or cream out for application to the patient. Further, other disclosed compositions of the present invention may be bottled or canned prior to use by a patient, subject or practitioner.

The composition containing H₂, or H₂ and O₂, can be formulated into a topical cream or gel wherein the cream or gel is applied topically and the H₂, or H₂ and O₂ is capable of transdermally penetrating the skin, mucosa or other surface to which the composition is applied.

Embodiments also include methods of treating ailments such as skin conditions using the compositions described herein. Such ailments can include, for example, psoriasis, acne (acne vulgaris), atopic dermatitis (eczema), shingles (herpes zoster), hives (urticaria), sunburn, contact dermatitis, diaper rash, rosacea, epidermolysis bullosa, hidradenitis suppurativa (HS), ichthyosis, pachyonychia congenita, pemphigus, and cancer (e.g., melanoma). In aspects, the compositions are applied directly to affected areas of skin. In aspects, the compositions described herein also include a rectally suitable carrier. In some embodiments, the compositions described herein are formulated as a suppository, an enema solution, a rectal foam, a rectal gel (i.e., for intrarectal administration) or vaginal solution/foam.

Dry Eye Disease symptoms include dryness in the eye, irritation, redness, discharge, blurred vision, and easily fatigued eyes. Symptoms range from mild and occasional to severe and continuous. Types of dry eye syndrome include evaporative, aqueous deficient, and mixed. This can be caused by age, contact lens use, meibomian gland dysfunction, pregnancy, Sjogren syndrome, vitamin A deficiency, omega-3 fatty acid deficiency, LASIK surgery, and certain medications such as antihistamines, some blood pressure medication, hormone replacement therapy, and antidepressants. All types of Dry Eye Disease have and underlying inflammatory etiology. Symptoms of dry eye syndrome can include one or more of: dryness, burning, a sandy-gritty eye irritation that gets worse as the day goes on, itchiness, stinging and/or tired eyes. Other symptoms can include one or more of pain, redness, a pulling sensation, and pressure behind the eye, which can manifest as a feeling that something, such as a speck of dirt, is in the eye. Dry eye syndrome can result in damage to the eye's surface which can result in an increase in discomfort and sensitivity to bright light. Dry eye can affect one eye, but in many instances, both eyes are affected. In an embodiment, dry eye is treated with a topically applied molecular hydrogen (H2) through administration of one or more eye drops, eye soak, or by mouth. In another embodiment, administration of molecular hydrogen (H2) mitigates the conditions associated with dry eye syndrome.

In a further embodiment, an individual suffering from dry eye is administered molecular hydrogen (H2) only to treat the syndrome. In an embodiment, an individual suffering from dry eye is administered molecular hydrogen (H2) and one or more of the following: a topical corticosteroid, cyclosporin, including ciclosporin A, diquafosol, lifitegrast, varenicline, n-acetylcystein, hyaluronic acid, rebamipide or perffluorohexyloctane. In another embodiment, molecular hydrogen (H2) is provided to an individual following surgery (tarsorrhyaphy) that is performed to reduce the eyelid separation.

Vascular endothelial growth factor (VEGF) has been associated with several diseases. For instance, VEGF after an injury to the central nervous system (CNS), VEGF expression is upregulated. VEGF expression is also associated with cancer growth. Other diseases that are associated with VEGF expression and/or high serum levels of VEGF include stroke, ischemic stroke, rheumatoid arthritis, diabetic retinopathy, age-related macular degeneration, angiosarcoma, lymphangioleiomyomatosis, glomeruli, pre-eclampsia.

In an embodiment, an individual suffering from a disease that results from expression, including upregulation of VEGF, is administered molecular hydrogen (H2) to reduce the production of VEGF.

Administration of molecular hydrogen (H2) to an individual reduces the production of VEGF by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to an individual not receiving the same treatment. In another embodiment, administration of molecular hydrogen (H2) reduces the production of VEGF by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to an individual not receiving the same treatment.

Administration of molecular hydrogen (H2) to an individual reduces the production of VEGF by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% as compared to an individual not receiving the same treatment.

Administration of molecular hydrogen (H2) to an individual reduces the severity of a disease that results from an increase in the production of VEGF by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to an individual not receiving the same treatment. In another embodiment, administration of molecular hydrogen (H2) reduces the severity of a disease that results from an increase in the production of VEGF by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to an individual not receiving the same treatment. Administration of molecular hydrogen (H2) to an individual reduces the severity of a disease that results from an increase in the production of VEGF by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% as compared to an individual not receiving the same treatment.

In an embodiment, the molecular hydrogen (H2) is administered to an individual by one or more of: intravenous infusion, orally, subcutaneously, intramuscularly, intranasally, intradermally, intramuscularly, intra anally, intravaginally or into an organ itself. Administration of molecular hydrogen (H2) reduces the development and progression of a disease that is characterized and/or results from increased angiogenesis.

In an embodiment, administration of molecular hydrogen (H2) is through the administration of a magnesium pellet that is inserted into the body. The magnesium pellet results in the generation of molecular hydrogen (H2). Administration of a magnesium pellet can be used as a long term treatment for a diseases that results from the increased production of VEGF. In an embodiment, a magnesium pellet is administered under the skin, intracamerally, intravitreally, intraocularly, subcutaneously, intravenously or intramuscularly.

Corneal microcysts are a most-distinctive and easily detectable indicator of contact lens-induced hypoxia. These include microcystic edema corneal microcysts. In an embodiment, a corneal microcyst results from inflammation or apoptosis.

In an embodiment, molecular hydrogen (H2) is applied to the eye of an individual topically. Topical administration can include administration by a drop or an eye soak. In another embodiment, molecular hydrogen (H2) for the treatment of corneal micro-cysts is administered orally, intravenously, intravitreal, intranasally, subcutaneously, intramuscularly, intra anal and intradermally.

In an embodiment, administration of molecular hydrogen (H2) to the eye is able to reduce the number of micro-cysts. In a further embodiment the number of micro-cysts is reduced by 1, 2, 3, 4, 5, 6, 7, 8, 9, 10, 11, 12, 13, 14, 15, 16, 17, 18, 19, 20 or more following administration of molecular hydrogen (H2) to an individual. In an embodiment, the molecular hydrogen (H2) is administered to an eye of the individual.

In another embodiment, corneal micro-cysts occur in patients who have been administered an Antibody Drug Conjugates (ADC's). An ADC comprises an antibody linked to a biologically active cytotoxic or cytostatic payload or drug and can be sued to target a specific antigen. In an embodiment, the antigen is a tumor antigen. ADCs include gemtuzumab ozogamicin, brentuximab vedotin, trastuzumab emtansine, inotuzumab ozogamicin, polatuzumab vedotin, enfortumab vedotin, trastuzumab deruxtecan, Sacituzumab govitecan, benlantamab mafodotin, moxetumomab pasudotox, loncastimab tesirin, tisotumab vedotin-tftv, mirvetuximab soravtansin, datopotamab deruxtecan-dink and telisotuzumab vedotin. The ADC toxic payload can include monomethylauristatin E, monomethyl auristatin F, mertansine, calicheamicin, a topoisomerase inhibitor, glucocorticoid receptor modulators, dexamethasone, an siRNA and budesonide and others.

In an embodiment, molecular hydrogen (H2) is administered to an individual at the same time that the individual is administered an ADC. In another embodiment, molecular hydrogen (H2) is administered to an individual prior to or following administration of an ADC. In a further embodiment, molecular hydrogen (H2) is administered 1, 2, 3, 4, 5, 6, 7, 8, 9, 10,11, 12, 13, 14, 15, 16, 17, 18, 19, 20 or more times to an individual that is administered an ADC.

Other cyst-like syndromes that can be treated with molecular hydrogen (H2), including inclusions that occur, include Meesmann's dystrophy, bullous keratopathy, and Cogan's microcystic dystrophy, or with mucin balls, vacuoles, microcystic edema, and infiltrates.

Administration of molecular hydrogen (H2) to an individual suffering from a corneal micro-cyst reduces the number of micro-cysts by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to an individual not receiving the same treatment.

In another embodiment, administration of molecular hydrogen (H2) to an individual suffering from a corneal micro-cyst reduces the number of micro-cysts by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to an individual not receiving the same treatment.

Administration of molecular hydrogen (H2) to an individual suffering from a corneal micro-cyst reduces the number of micro-cysts by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% as compared to an individual not receiving the same treatment.

Administration of molecular hydrogen (H2) to an individual reduces the severity of a corneal micro-cyst by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to an individual not receiving the same treatment.

In another embodiment, administration of molecular hydrogen (H2) reduces the severity of a corneal micro-cyst by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to an individual not receiving the same treatment.

Administration of molecular hydrogen (H2) to an individual reduces the severity of a corneal micro-cyst by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% as compared to an individual not receiving the same treatment.

Inflammatory bowel disease (IBD) comprises several syndromes that result in an inflammatory condition of the colon and small intestine. IBD include Crohn's disease and ulcerative colitis (UC) as the principal IBD syndromes. Crohn's disease affects the small and large intestine, as well as the mouth, esophagus, stomach and the anus. Ulcerative colitis principally affects the colon and the rectum.

Current treatments for IBD include surgery, for example, proctocolectomy. In another embodiment, the treatment of IBD is through the administration of one or more drugs, including, immunosuppressive drugs. In a further embodiment, an individual suffering from an IBD is administered a steroid, such as prednisone. Other treatments include administration of one or more of tumor necrosis factor inhibitors, azathioprine, methotrexate or 6-mercaptopurine. Mesalazine is used to treat ulcerative colitis. Budesonide is used for mile Crohn's disease.

In an embodiment, molecular hydrogen (H2)_is administered to an individual suffering from an IBD. In another embodiment, molecular hydrogen (H2) administered to an individual suffering from an IBD where the individual is also treated with one or more other drugs.

In an embodiment, molecular hydrogen (H2) is administered to an individual orally, intravenously, intranasally, subcutaneously, intramuscularly, intraanally and intradermally.

Administration of molecular hydrogen (H2) to an individual suffering from an IBD reduces the severity of the IBD by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to an individual not receiving the same treatment.

In another embodiment, administration of molecular hydrogen (H2) to an individual suffering from IBD reduces the severity of the IBD by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to an individual not receiving the same treatment.

Administration of molecular hydrogen (H2) to an individual suffering from IBD reduces the severity of the IBD by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% as compared to an individual not receiving the same treatment.

Administration of molecular hydrogen (H2) to an individual suffering from IBD results in a reduction in the symptoms of an IBD by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to an individual not receiving the same treatment.

In another embodiment, administration of molecular hydrogen (H2) to an individual suffering from IBD results in a reduction in the symptoms of an IBD by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to an individual not receiving the same treatment.

Administration of molecular hydrogen (H2) to an individual an individual suffering from IBD results in a reduction in the symptoms of an IBD by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% as compared to an individual not receiving the same treatment.

Psoriasis is noncontagious autoimmune disease that can be long lasting and is characterized by patches of abnormal skin that are generally red, pink, or purple in color. Psoriatic patches are also subject to being dry, itchy, and in more severe cases, scaly. Psoriasis varies in severity from small localized patches on specific parts of the body like the arms, legs and torso to covering the whole body. Psoriasis can be triggered by multiple factors, including injury to the skin, a phenomena known as the Koebner phenomenon.

There are five main types of psoriasis. These include plaque, guttate, inverse, pustular and erythrodermic. Plaque psoriasis, also known as psoriasis vulgaris, makes up about 90% of psoriasis cases. Treatment of psoriasis generally involves the use one or more drugs, including corticosteroids, ditrhinol, fluocinonide, ciclosporin, hydroxycarbamide, fumarates, dimethyl fumarate, methotrexate, anti-TNF therapies, infliximab, adalimumab, glimumab, certolizumab pegol, apremilast, efalizumab, alefacept, etanercept, bimekizumab, ixekizumab, Risankizumab, apremilast, antistreptococcal medications, vitamin D3 analogues, retinoids, vitamin D analogues, such as paricalcitol, calcipotriol, tacalcitol and calcitriol and coal tar. Other treatments for psoriasis include mineral oil, sialic acid, petroleum jelly and decubal. A further treatment for psoriasis is phototherapy, using the sun or a UV-B lamp and removal of the tonsils. Psoriasis can also be treated with an antibody, a chimeric antigen receptor (CAR) or an ADC that kills the immune cells responsible for causing the psoriasis.

In an embodiment, psoriasis is treated with molecular hydrogen (H2). In another embodiment, psoriasis is treated with molecular hydrogen (H2) and one or more other drugs. In a further embodiment, psoriasis is treated with molecular hydrogen (H2) and phototherapy, one or more drugs and/or removal of the tonsils. In an embodiment, psoriasis is treated with molecular hydrogen (H2) and an antibody, a chimeric antigen receptor (CAR) or an ADC that kills the immune cells responsible for causing the psoriasis.

Molecular hydrogen is administered to an individual topically, orally, intravenously, intranasally, subcutaneously, intramuscularly, intravitreally, intra anally and intradermally. In another embodiment, molecular hydrogen (H2) is administered using a magnesium pellet.

In an embodiment, a pellet can be a sphere, a square, an ovoid, a rectangular share, an amorphous shape or any shape that is known to one of skill in the art.

Administration of molecular hydrogen (H2) to an individual suffering from a psoriasis reduces the severity of the psoriasis by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to an individual not receiving the same treatment.

In another embodiment, administration of molecular hydrogen (H2) to an individual suffering from a psoriasis reduces the severity of the psoriasis by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to an individual not receiving the same treatment.

Administration of molecular hydrogen (H2) to an individual suffering from a psoriasis reduces the severity of the psoriasis by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% as compared to an individual not receiving the same treatment.

Administration of molecular hydrogen (H2) to an individual reduces the number of psoriatic plaques on the individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to an individual not receiving the same treatment.

In another embodiment, administration of molecular hydrogen (H2) reduces the number of psoriatic plaques on the individual by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to an individual not receiving the same treatment.

Administration of molecular hydrogen (H2) to an individual reduces the number of psoriatic plaques on the individual by at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100% as compared to an individual not receiving the same treatment.

Additional embodiments include methods of treating oral ailments using the compositions described herein. In aspects, the compositions can be used as an oral rinse. Oral ailments can include, for example, premalignant or precancerous (also referred to as “potentially malignant”) oral lesions, cavities (tooth decay), gum (periodontal) disease, cold sores, denture stomatitis, dry mouth, geographic tongue, fungal/yeast growth, human papilloma virus (HPV), ulcers and oral cancer. In aspects, sodium bicarbonate is added to induce foaming and/or sucralose is added to improve taste. In aspects, the compositions described herein are formulated for intranasal administration or oromucosal administration.

Formulations provided herein are also used in methods of treating a cancer or tumor, including, for instance, retinoblastoma, neck cancer, laryngeal cancer and/or melanoma.

The compositions of the present invention may be a formulation of H₂:O₂ at a 1:1 ratio, a 1.1:1 ratio, a 1.2:1 ratio, a 1.3:1 ratio, a 1.4:1 ratio, a 1.5:1 ratio, a 1.6:1 ratio, a 1.7:1 ratio, a 1.8:1 ratio, a 1.9:1 ratio, a 2:1 ratio, a 2.1:1 ratio, a 2.2:1 ratio, a 2.3:1 ratio, a 2.4:1 ratio, a 2.5:1 ratio, a 2.6:1 ratio, a 2.7:1 ratio, a 2.8:1 ratio, a 2.9:1 ratio, a 3:1 ratio, a 3.1:1 ratio, a 3.2:1 ratio, a 3.3:1 ratio, a 3.4:1 ratio, a 3.5:1 ratio, a 3.6:1 ratio, a 3.7:1 ratio, a 3.8:1 ratio, a 3.9:1 ratio, a 4:1 ratio, a 5:1 ratio, a 6:1 ratio, a 7:1 ratio, an 8:1 ratio, a 9:1 ratio or a 10:1 ratio.

In embodiments, the compositions and delivery methods of the present invention reduce signs and symptoms of red eye by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of red eye by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%. 163. In embodiments, the compositions and delivery methods of the present invention reduce signs and symptoms of red eye by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of dry eye disease by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of dry eye disease by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of dry eye disease by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of an eye disease or infection by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of an eye disease or infection by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of dry eye disease by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of an eye disease or infection by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of an allergy by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of an allergy by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of allergies by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of post-operative inflammation by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of post-operative inflammation by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of post-operative inflammation by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of iritis by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of iritis by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of iritis by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of uveitis by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of uveitis by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of uveitis by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of scleritis by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of scleritis by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of scleritis by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of episcleritis by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of episcleritis by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of episcleritis by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of chemical burns by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of chemical burns by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of chemical burns by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of inflammation associated with infection by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of inflammation associated with infection by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of inflammation associated with infection by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of cystoid macular edema by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of cystoid macular edema by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of cystoid macular edema by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of giant papillary conjunctivitis by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of giant papillary conjunctivitis by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of giant papillary conjunctivitis by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of medicamentosa by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of medicamentosa by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of medicamentosa by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.In embodiments, the compositions and delivery methods of the present invention reduce signs and symptoms of central serous chorioretinopathy by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of central serous chorioretinopathy by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of central serous chorioretinopathy by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of central serous chorioretinopathy by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

In embodiments, the compositions and delivery methods of the present invention reduce signs and symptoms of macular edema by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and delivery methods of the present invention reduce signs and symptoms of macular edema by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%.

In embodiments, the compositions and delivery methods of the present invention reduce signs and symptoms of macular edema by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.In embodiments, the compositions and delivery methods of the present invention reduce signs and symptoms of ocular sequela by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%.

In embodiments, the compositions and the methods of delivery methods of the present invention of the composition reduce signs and symptoms of ocular sequela by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the composition delivery methods of the present invention reduce signs and symptoms of ocular sequela by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the composition delivery methods of the present invention reduce signs and symptoms of ocular sequela by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the composition delivery methods of the present invention reduce signs and symptoms of contact-lens-related irritation by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the composition reduce signs and symptoms of contact-lens-related irritation by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the composition reduce signs and symptoms of contact-lens-related irritation by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.In embodiments, the compositions and delivery methods of the present invention reduce signs and symptoms of physical damage resulting from a previous ocular treatment by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the composition reduce signs and symptoms of physical damage resulting from a previous ocular treatment by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99 compared to an individual that is not administered the compositions In embodiments, the compositions and the methods of delivery of the composition reduce signs and symptoms of physical damage resulting from a previous ocular treatment by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the composition reduce signs and symptoms of physical damage resulting from a previous ocular treatment by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the composition reduce signs and symptoms of retinal blood vessel blockage by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the composition reduce signs and symptoms of retinal blood vessel blockage, including blockage of a vein or artery, by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the composition reduce signs and symptoms of retinal blood vessel blockage by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the composition reduce signs and symptoms of blurred vision by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the composition reduce signs and symptoms of blurred vision by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the composition reduce signs and symptoms of blurred vision by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the composition reduce the size of corneal micro cysts by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the composition reduce the size of corneal micro cysts by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the composition reduce the size of corneal microcysts by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduce protein deposits in a subject's or patient's eye by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce protein deposits in a subject's or patient's eye by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce protein deposits in a subject's or patient's eye by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduce protein deposits under the macula of a subject's or patient's eye by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce protein deposits under the macula of a subject's or patient's eye by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce protein deposits under the macula of a subject's or patient's eye by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduce protein deposits on the macula of a subject's or patient's eye by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce protein deposits on the macula of a subject's or patient's eye by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce protein deposits on the macula of a subject's or patient's eye by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduce the prevalence of corneal micro cysts by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce the prevalence of corneal micro cysts by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce the prevalence of corneal micro cysts by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduces collagen fiber content in a patient's or subject's vitreous by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduces collagen fiber content in a patient's or subject's vitreous by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduces collagen fiber content in a patient's or subject's vitreous by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduce the surface area of iritis lesions by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce the surface area of iritis lesions by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce the surface area of iritis lesions by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduce the surface area of uveitis lesions by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce the surface area of uveitis lesions by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce the surface area of uveitis lesions by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduce the surface area of scleritis lesions by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce the surface area of scleritis lesions by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce the surface area of scleritis lesions by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduce the surface area of episcleritis lesions by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce the surface area of episcleritis lesions by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce the surface area of episcleritis lesions by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduce the surface area of red eye by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce the surface area of red eye by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce the surface area of red eye by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99%.

compared to an individual that is not administered the compositions at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduce the surface area of post-injury inflammation by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduce the surface area of post-injury inflammation by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions improves retinal function by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions improves retinal function by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions improves retinal function by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions increases field of vision in a patient or subject by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions increases field of vision in a patient or subject by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions increases field of vision in a patient or subject by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions restores central vision by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions restores central vision by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions restores central vision by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduces the volume of fluid under a patient's or subject's retina by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduces the volume of fluid under a patient or subject by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduces the volume of fluid under a patient or subjects retina by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduces the perceived instances of vitreous floaters by a patient or subject by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduces the perceived instances of vitreous floaters by a patient or subject by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduces the perceived instances of vitreous floaters by a patient or subject by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduces the presence of vascular endothelial growth factor (VEGF) by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduces the presence of vascular endothelial growth factor (VEGF) by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

In embodiments, the compositions and the methods of delivery of the compositions reduces the presence of vascular endothelial growth factor (VEGF) by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In embodiments, the compositions and the methods of delivery of the compositions reduces scarring of the macula by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% compared to an individual that is not administered the compositions.

In embodiments, the compositions and the methods of delivery of the compositions reduces the scarring of the macula by e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% compared to an individual that is not administered the compositions.

the methods of delivery of the compositions and the methods of the present invention reduces the scarring of the macular by e.g., no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 96%, no more than 97%, no more than 98%, no more than 99% compared to an individual that is not administered the compositions.

In an aspect, the hydrogen gas of the composition of the instant invention is present at an amount of at least 0.1 ppm, at least 0.2 ppm, at least 0.3 ppm, at least 0.4 ppm, at least 0.5 ppm, at least 0.6 ppm, at least 0.7 ppm, at least 0.8 ppm, at least 0.9 ppm, at least 1 ppm, at least 1.2 ppm, at least 1.5 ppm, at least 1.75 ppm, at least 2 ppm, at least 2.25 ppm, at least 2.5 ppm, at least 2.75 ppm, at least 3 ppm, at least 4 ppm, at least 5 ppm, at least 6 ppm, at least 7 ppm, at least 8 ppm, at least 9 ppm, at least 10 ppm, at least 11 ppm, at least 12 ppm, at least 13 ppm, at least 14 ppm, at least 15 ppm, at least 16 ppm, at least 17 ppm, at least 18 ppm, at least 19 ppm, at least 20 ppm, at least 21 ppm, at least 25 ppm, at least 30 ppm, at least 35 ppm, at least 40 ppm, at least 45 ppm, at least 50 ppm, at least 60 ppm, at least 75 ppm, at least 100 ppm, or more.

In aspects, the hydrogen gas of the composition of the instant invention is present at an amount of no more than 0.1 ppm, no more than 0.2 ppm, no more than 0.3 ppm, no more than 0.4 ppm, no more than 0.5 ppm, no more than 0.6 ppm, no more than 0.7 ppm, no more than 0.8 ppm, no more than 0.9 ppm, no more than 1 ppm, no more than 1.2 ppm, no more than 1.5 ppm, no more than 1.75 ppm, no more than 2 ppm, no more than 2.25 ppm, no more than 2.5 ppm, no more than 2.75 ppm, no more than 3 ppm, no more than 4 ppm, no more than 5 ppm, no more than 6 ppm, no more than 7 ppm, no more than 8 ppm, no more than 9 ppm, no more than 10 ppm, no more than 11 ppm, no more than 12 ppm, no more than 13 ppm, no more than 14 ppm, no more than 15 ppm, no more than 16 ppm, no more than 17 ppm, no more than 18 ppm, no more than 19 ppm, no more than 20 ppm, no more than 21 ppm, no more than 25 ppm, no more than 30 ppm, no more than 35 ppm, no more than 40 ppm, no more than 45 ppm, no more than 50 ppm, no more than 60 ppm, no more than 75 ppm, or no more than 100 ppm.

In aspects, the composition of the present invention comprises an oxygen gas concentration of at least 7 mg/L, at least 7.5 mg/L, at least 8 mg/L, at least 8.5 mg/L, at least 9 mg/L, at least 9.25 mg/L, at least 9.5 mg/L, at least 9.75 mg/L, at least 10 mg/L, at least 10.25 mg/L, at least 10.5 mg/L, at least 10.75 mg/L, at least 11 mg/L, at least 11.25 mg/L, at least 11.5 mg/L, at least 11.75 mg/L, at least 12 mg/L, at least 12.25 mg/L, at least 12.5 mg/L, at least 12.75 mg/L, at least 13 mg/L, at least 13.25 mg/L, at least 13.5 mg/L, at least 13.75 mg/L, at least 14 mg/L, at least 14.5 mg/L, at least 15 mg/L, at least 16 mg/L, at least 17 mg/L, at least 18 mg/L, at least 19 mg/L, at least 20 mg/L, at least 21 mg/L, at least 25 mg/L, at least 30 mg/L, at least 40 mg/L, at least 45 mg/L, at least 50 mg/L, at least 75 mg/L, at least 100 mg/L or more.

In aspects, the composition of the present invention comprises an oxygen gas concentration of no more than 7 mg/L, no more than 7.5 mg/L, no more than 8 mg/L, no more than 8.5 mg/L, no more than 9 mg/L, no more than 9.25 mg/L, no more than 9.5 mg/L, no more than 9.75 mg/L, no more than 10 mg/L, no more than 10.25 mg/L, no more than 10.5 mg/L, no more than 10.75 mg/L, no more than 11 mg/L, no more than 11.25 mg/L, no more than 11.5 mg/L, no more than 11.75 mg/L, no more than 12 mg/L, no more than 12.25 mg/L, no more than 12.5 mg/L, no more than 12.75 mg/L, no more than 13 mg/L, no more than 13.25 mg/L, no more than 13.5 mg/L, no more than 13.75 mg/L, no more than 14 mg/L, no more than 14.5 mg/L, no more than 15 mg/L, no more than 16 mg/L, no more than 17 mg/L, no more than 18 mg/L, no more than 19 mg/L, no more than 20 mg/L, no more than 21 mg/L, no more than 25 mg/L, no more than 30 mg/L, no more than 40 mg/L, no more than 45 mg/L, no more than 50 mg/L, no more than 75 mg/L, no more than 100 mg/L.

In aspects, the composition of the present invention comprises an oxygen gas concentration of about 7 mg/L, about 7.5 mg/L, about 8 mg/L, about 8.5 mg/L, about 9 mg/L, about 9.25 mg/L, about 9.5 mg/L, about 9.75 mg/L, about 10 mg/L, about 10.25 mg/L, about 10.5 mg/L, about 10.75 mg/L, about 11 mg/L, about 11.25 mg/L, about 11.5 mg/L, about 11.75 mg/L, about 12 mg/L, about 12.25 mg/L, about 12.5 mg/L, about 12.75 mg/L, about 13 mg/L, about 13.25 mg/L, about 13.5 mg/L, about 13.75 mg/L, about 14 mg/L, about 14.5 mg/L, about 15 mg/L, about 16 mg/L, about 17 mg/L, about 18 mg/L, about 19 mg/L, about 20 mg/L, about 21 mg/L, about 25 mg/L, about 30 mg/L, about 40 mg/L, about 45 mg/L, about 50 mg/L, about 75 mg/L, about 100 mg/L.

In aspects, the composition of the present invention comprises a liquid oxygen concentration of at least 7 mg/L, at least 7.5 mg/L, at least 8 mg/L, at least 8.5 mg/L, at least 9 mg/L, at least 9.25 mg/L, at least 9.5 mg/L, at least 9.75 mg/L, at least 10 mg/L, at least 10.25 mg/L, at least 10.5 mg/L, at least 10.75 mg/L, at least 11 mg/L, at least 11.25 mg/L, at least 11.5 mg/L, at least 11.75 mg/L, at least 12 mg/L, at least 12.25 mg/L, at least 12.5 mg/L, at least 12.75 mg/L, at least 13 mg/L, at least 13.25 mg/L, at least 13.5 mg/L, at least 13.75 mg/L, at least 14 mg/L, at least 14.5 mg/L, at least 15 mg/L, at least 16 mg/L, at least 17 mg/L, at least 18 mg/L, at least 19 mg/L, at least 20 mg/L, at least 21 mg/L, at least 25 mg/L, at least 30 mg/L, at least 40 mg/L, at least 45 mg/L, at least 50 mg/L, at least 75 mg/L, at least 100 mg/L or more.

In aspects, the composition of the present invention comprises a liquid oxygen concentration of no more than 7 mg/L, no more than 7.5 mg/L, no more than 8 mg/L, no more than 8.5 mg/L, no more than 9 mg/L, no more than 9.25 mg/L, no more than 9.5 mg/L, no more than 9.75 mg/L, no more than 10 mg/L, no more than 10.25 mg/L, no more than 10.5 mg/L, no more than 10.75 mg/L, no more than 11 mg/L, no more than 11.25 mg/L, no more than 11.5 mg/L, no more than 11.75 mg/L, no more than 12 mg/L, no more than 12.25 mg/L, no more than 12.5 mg/L, no more than 12.75 mg/L, no more than 13 mg/L, no more than 13.25 mg/L, no more than 13.5 mg/L, no more than 13.75 mg/L, no more than 14 mg/L, no more than 14.5 mg/L, no more than 15 mg/L, no more than 16 mg/L, no more than 17 mg/L, no more than 18 mg/L, no more than 19 mg/L, no more than 20 mg/L, no more than 21 mg/L, no more than 25 mg/L, no more than 30 mg/L, no more than 40 mg/L, no more than 45 mg/L, no more than 50 mg/L, no more than 75 mg/L, no more than 100 mg/L.

In aspects, the composition of the present invention comprises a liquid oxygen concentration of about 7 mg/L, about 7.5 mg/L, about 8 mg/L, about 8.5 mg/L, about 9 mg/L, about 9.25 mg/L, about 9.5 mg/L, about 9.75 mg/L, about 10 mg/L, about 10.25 mg/L, about 10.5 mg/L, about 10.75 mg/L, about 11 mg/L, about 11.25 mg/L, about 11.5 mg/L, about 11.75 mg/L, about 12 mg/L, about 12.25 mg/L, about 12.5 mg/L, about 12.75 mg/L, about 13 mg/L, about 13.25 mg/L, about 13.5 mg/L, about 13.75 mg/L, about 14 mg/L, about 14.5 mg/L, about 15 mg/L, about 16 mg/L, about 17 mg/L, about 18 mg/L, about 19 mg/L, about 20 mg/L, about 21 mg/L, about 25 mg/L, about 30 mg/L, about 40 mg/L, about 45 mg/L, about 50 mg/L, about 75 mg/L, about 100 mg/L.

Alternatively, use of the compositions provided herein (i.e., compositions of Table 1) for treating or healing as described herein may be by topical administration such as a drop (i.e., ocular instillation), an ointment, a soak, a gas, a gel, a cream, a mist, or a spray. In some embodiments, the compositions are used on an eye of a subject. The subject may be a human, or another animal. In some embodiments, the animal is a domesticated animal, including livestock, or a pet.

As an additional aspect, the invention includes kits which comprise a composition of the invention packaged in a manner which facilitates its use for administration to subjects. In one embodiment, such a kit includes a compound or composition described herein, packaged in a container such as a sealed bottle, container, tube or other vessel, with a label affixed to the container or included in the package that describes use of the compound or composition in practicing the method. In one embodiment, the kit contains a first container having a composition of the present invention and a second container having a physiologically acceptable reconstitution solution for the composition in the first container. In one aspect, the compound or composition is packaged in a unit dosage form. The kit may further include a device suitable for administering the composition according to a specific route of administration. Preferably, the kit contains a label that describes use of a therapeutic protein or peptide composition.

Dosing can be single dosage or cumulative (serial dosing), and can be readily determined by one skilled in the art. For instance, treatment of a disease, infection or syndrome may comprise a one-time administration of an effective dose of a composition disclosed herein. Alternatively, treatment of a disease, infection or syndrome may comprise multiple administrations of an effective dose of a composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a composition disclosed herein that is administered can be adjusted accordingly.

In a further embodiment, composition disclosed herein have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a composition disclosed herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period during which administration of the composition disclosed herein is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Single or multiple administrations of the compositions can be carried out with the dose levels and pattern being selected by the treating physician. For the prevention or treatment of disease, the appropriate dosage will depend on the type of disease to be treated, as described above, the severity and course of the disease, whether the composition is being administered for preventive or therapeutic purposes, previous therapy, the patient's clinical history and response to the composition, and the discretion of the attending physician.

In an embodiment, a sustained release delivery composition releases a the composition over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In another embodiment, a sustained release delivery composition releases the composition disclosed herein with substantially zero order release kinetics over a period, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

In an embodiment, a composition is in the form of a long acting composition that includes, without limitation, extended release compositions. An embodiment includes, without limitation, an extended release liquid, gel, cream, a semi-solid or solid formulation that provides the composition to the patient to whom it is administered over time. The long acting composition can provide the H₂ or H₂ and O₂ in a patient for 4 hours, 6 hours, 8 hours, 10 hours, 12 hours, 16 hours, 20 hours, 24 hours, 28 hours, 30 hours, 32 hours, 34 hours, 36 hours, 40 hours, 48 hours, 3 days, 4 days, 5 days, 6 days, 7 days, 2 weeks, 3 weeks or 4 weeks. The long acting formulations can provide activity of a composition for as little as 4 hours or as long as 4 weeks, for as little as 4 hours or as long as 3 weeks, for as little as 4 hours or as long as 2 weeks, for as little as 4 hours or as long as 1 week, for as little as 4 hours or as long as 6 days, for as little as 4 hours or as long as 5 days, for as little as 4 hours or as long as 4 days, for as little as 4 hours or as long as 3 days, for as little as 4 hours or as long as 2 days, for as little as 4 hours or as long as 1 day, for as little as 4 hours or as long as 20 hours, for as little as 4 hours or as long as 16 hours, for as little as 4 hours or as long as 14 hours, for as little as 4 hours or as long as 12 hours, for as little as 4 hours or as long as 10 hours, for as little as 4 hours or as long as 8 hours, for as little as 4 hours or as long as 6 hours.

In an embodiment, a sustained release composition releases a H₂ or H₂ and O₂ disclosed herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release composition releases a H₂ or H₂ and O₂ disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

In an embodiment, a composition releases a H₂ or H₂ and O₂ disclosed herein over a period of, without limitation, about 1 day after administration, about 2 days after administration, about 3 days after administration, about 4 days after administration, about 5 days after administration, about 6 days after administration or about 7 days or more after administration. In an additional embodiment, a composition releases a H₂ or H₂ and O₂ disclosed herein with substantially zero order release kinetics over a period of, without limitation, at most 1 day after administration, at most 2 days after administration, at most 3 days after administration, at most 4 days after administration, at most 5 days after administration, at most 6 days after administration or at most 7 days or more after administration.

In an embodiment, a composition releases a H₂ or H₂ and O₂ disclosed herein with release kinetics over a period of, without limitation, about 1 day after administration, about 2 days after administration, about 3 days after administration, about 4 days after administration, about 5 days after administration, about 6 days after administration or about 7 days or more after administration. In an additional embodiment, a composition releases a H₂ or H₂ and O₂ disclosed herein with substantially first order release kinetics over a period of, e.g., at most 1 day after administration, at most 2 days after administration, at most 3 days after administration, at most 4 days after administration, at most 5 days after administration, at most 6 days after administration or at most 7 days or more after administration.

### EXAMPLES

### Example 1: Assessment of a Hydrogen/Oxygen Solution and a Hydrogen Solution on Irritated Rabbit Corneas

Topical 0.1% benzalkonium chloride (BAC) are administered to the eyes of male New Zealand white rabbits as a model for induced ocular irritation, as described in Table 2 (below). In ophthalmology, stains are applied to the surface of the eye and examined with a microscope to evaluate and quantify the extent of dryness or other ocular pathology. The industry standard for assessing the severity of dry eye syndrome is lissamine green stain. Fluorescein stain is also widely used.

Table 2 shows the study design. Four rabbits are in each of the three groups; each group's dosing route being topical ocular. Group 1 is the control group with BAC challenge, and sterile water soak. Group 2 receives the BAC challenge, and HHO (composition 8) soak. Group 3 receives the BAC challenge, and H₂ (composition 1) soak.

A total of 20 animals are administered BAC OD on days 1 through 24 of the study. The BAC OD is administered three times daily, approximately 4 hours apart, for 14 days (days 1 through 14), followed by once daily for 10 days. Following the Day 24 ophthalmic exam, 12 animals were selected for study based upon conditioning, ophthalmic findings, and staining scores. All doses were administered at approximately the same time each day±5 minutes.

### Legend:

BAC (benzalkonium chloride); OD (right eye); OU (both eyes); SDW (sterile distilled water); HHO (hydrogen and oxygen solution; composition 8); H₂, Hydrogen solution (composition 1); NA (not applicable);
(a) administered three times daily, approximately 4 hours apart, for 14 days (days 1 through 14), followed by once daily for 24 days (days 16 through 37 in the a.m.), as a 0.1% solution to generate a dry eye condition;
(b) administered once daily beginning on day 25 for 13 days (days 25 through 37)—the dose was in the P.M. after the BAC dose for days 25 through 37; and
(c) administered four times daily, approximately 2 hours apart, beginning on day 25 for 13 days (days 25 through 37)—the HHO (group 2) or H₂ (group 3) doses were after the BAC dose for days 25 through 37.

TABLE 2
Study design

| | | | Target Dose | Target Dose | Sample |
|---|---|---|---|---|---|
| | | | Level | Volume | Collec- |
| Group | Days | Treatment | (mg/eye/dose) | (μL/eye) | tions |
| 1 | 1-37 | BAC OD^{a} | 0.02 | 20 | Tears, |
| | 25-37 | SDW OU^{b} | NA | 500 | Eyes |
| 2 | 1-37 | BAC OD^{a} | 0.02 | 20 | Tears, |
| | 25-37 | HHO OU^{c} | NA | 500 | Eyes |
| 3 | 1-37 | BAC Od^{a} | 0.02 | 20 | Tears, |
| | 25-37 | H₂ OU^{c} | NA | 500 | Eyes |

Topical administration of 0.1% BAC to the right eyes resulted in a notable and prolonged increase in corneal fluorescein and lissamine green staining scores. In the left eyes (which were not administered BAC) corneal fluorescein and lissamine green staining scores remained relatively consistent between days 2 and 36, with the exception of day 21, when scores were higher for an undeterminable reason. This stability of staining scores during administration of the vehicle and both test articles indicates that topical administration of sterile distilled water, HHO, or H₂ is well tolerated.

For the right eyes of Group 1 animals administered 0.1% BAC and SDW (starting on day 25), corneal fluorescein and lissamine green staining scores remained relatively consistent between days 6 and 36, which indicates that the model system is stable during the dosing phase (days 25 through 36), and no reduction in staining scores attributable to SDW occurred. Topical application of HHO and H₂ resulted in a mean reduction in corneal fluorescein and lissamine green staining scores between days 28 and 36 in the right eyes treated with BAC. Combining data over the dosing phase and comparing the groups to each other during the dosing phase better controls for some of the day-to-day variability in staining scores. When the staining scores for the right eyes from each group are averaged over the entirety of the dosing phase examinations (days 28 through 36), mean corneal fluorescein and lissamine green staining scores for Group 1 administered SDW are 3.83 and 2.17, respectively. These are compared to scores of 3.00 and 1.58 for fluorescein and lissamine green staining, respectively, for the eyes administered HHO and 2.08 and 0.83 for eyes administered H₂.

These differences in overall mean scores suggest a clear treatment and efficacy effect for H₂ and a treatment and efficacy effect for HHO. While eyes treated with HHO and H₂ have mean lissamine and fluorescein staining scores that are much better than the control eyes, staining from day 36, the last day of the study, are indisputable. Cumulative staining scores of eyes in the control group stained with lissamine green is 10. Cumulative staining scores of all eyes treated with H₂ is 1. In other words, eyes treated with H₂ have 10× less staining than the control eyes. The results are shown in Tables 3-6.

TABLE 3
Mean Scores for Corneal Fluorescein Staining-Left Eye (Control);
Dosing of SDW, HHO, and H₂ Started on Day 25 (N = 4 eyes)

| | Day of Ophthalmic Examination | | | | | | |
|---|---|---|---|---|---|---|---|
| | 0 | 2 | 6 | 8 | 10 | 13 | 15 |
| Group 1 SDW | 0.25 | 1.25 | 1.5 | 3 | 1.25 | 1.25 | 1 |
| Group 2 HHO | 0.25 | 1.5 | 1.5 | 2 | 0.5 | 1.25 | 1.25 |
| Group 3 H₂ | 0.5 | 1.25 | 0.75 | 1 | 1 | 1 | 0.75 |

| | Day of Ophthalmic Examination | | | | | |
|---|---|---|---|---|---|---|
| | 17 | 21 | 24 | 28 | 31 | 36 |
| Group 1 SDW | 0.75 | 2.75 | 0.5 | 1.25 | 1 | 1.25 |
| Group 2 HHO | 0 | 2.25 | 0 | 0.25 | 1 | 1.25 |
| Group 3 H₂ | 0.5 | 1.75 | 0.25 | 0 | 0.75 | 0.25 |

TABLE 4
Mean Scores for Corneal Fluorescein Staining-Right Eye;
BAC Administration Started After Day O Examination, and Dosing
of SDW, HHO, and H₂ Started on Day 25 (N = 4 eyes)

| | Day of Ophthalmic Examination | | | | | | |
|---|---|---|---|---|---|---|---|
| | 0 | 2 | 6 | 8 | 10 | 13 | 15 |
| Group 1 BAC + SDW | 0.25 | 2.75 | 3.5 | 4.25 | 4.25 | 5 | 4.5 |
| Group 2 BAC + HHO | 0.25 | 2 | 2.75 | 3.25 | 3.5 | 3.75 | 3.75 |
| Group 3 BAC + H₂ | 0.25 | 2.25 | 2.25 | 2.5 | 3.75 | 3.5 | 3.25 |

| | Day of Ophthalmic Examination | | | | | |
|---|---|---|---|---|---|---|
| | 17 | 21 | 24 | 28 | 31 | 36 |
| Group 1 BAC + SDW | 3.25 | 4 | 2.75 | 3.75 | 3.75 | 4 |
| Group 2 BAC + HHO | 3.5 | 3.25 | 2.5 | 3.25 | 3 | 2.75 |
| Group 3 BAC + H₂ | 4 | 3.5 | 2.5 | 1.75 | 2.25 | 2 |

TABLE 5
Mean Scores for Lissamine Green Staining-Left Eye; Dosing
of SDW, HHO, and H₂ Started on Day 25 (N = 4 eyes)

| | Day of Ophthalmic Examination | | | | | | |
|---|---|---|---|---|---|---|---|
| | 0 | 2 | 6 | 8 | 10 | 13 | 15 |
| Group 1 SDW | 0 | 0 | 0.25 | 0.5 | 0.5 | 0.5 | 0.25 |
| Group 2 HHO | 0 | 0 | 0 | 0.75 | 0 | 0 | 0.5 |
| Group 3 H₂ | 0 | 0 | 0.5 | 0.25 | 0 | 0 | 0 |

| | Day of Ophthalmic Examination | | | | | |
|---|---|---|---|---|---|---|
| | 17 | 21 | 24 | 28 | 31 | 36 |
| Group 1 SDW | 0.25 | 1.75 | 0.75 | 0.5 | 0.25 | 0.25 |
| Group 2 HHO | 0 | 1 | 0 | 0.5 | 0.25 | 0.5 |
| Group 3 H₂ | 0 | 1.25 | 0.25 | 0 | 0.25 | 0 |

TABLE 6
Mean Scores for Corneal Lissamine Green Staining-Right Eye;
BAC Administration Started After Day O Examination, and Dosing
of SDW, HHO, and H₂ Started on Day 25 (N = 4 eyes)

| | Day of Ophthalmic Examination | | | | | | |
|---|---|---|---|---|---|---|---|
| | 0 | 2 | 6 | 8 | 10 | 13 | 15 |
| Group 1 BAC + SDW | 0 | 0.25 | 2.75 | 2.25 | 2.25 | 3.25 | 2 |
| Group 2 BAC + HHO | 0 | 0 | 1.5 | 2 | 1 | 1.5 | 1.75 |
| Group 3 BAC + H₂ | 0 | 0 | 1 | 0.5 | 0.75 | 0.5 | 2.25 |

| | Day of Ophthalmic Examination | | | | | |
|---|---|---|---|---|---|---|
| | 17 | 21 | 24 | 28 | 31 | 36 |
| Group 1 BAC + SDW | 1.25 | 1.75 | 1.75 | 2 | 2.25 | 2.5 |
| Group 2 BAC + HHO | 1.5 | 2.0 | 1.0 | 1.75 | 1.5 | 1.5 |
| Group 3 BAC + H₂ | 1.5 | 2.25 | 1.0 | 0.5 | 1.25 | 0.25 |

### Example 2: Treatment of Patient with Dry Eye

A 27-year-old patient visits an optometrist complaining of symptoms of dry eye from contact lens overuse. After a routine examination, the optometrist applies a composition of the present invention comprising molecular hydrogen (H₂) to the naked eyes of the patient.

The optometrist performs this procedure using a container containing a solution of molecular hydrogen and a buffer. The container is sealed by a quick-release seal at one end. The optometrist applies the quick-release seal to a naked eye of the patient and actuates the quick-release seal, effectively bathing the naked eye of the patient with molecular hydrogen solution. The optometrist repeats the procedure for the patient's other eye.

The optometrist gives the patient the container for personal use and requests that the patient bathe their naked eyes in the molecular hydrogen solution nightly. A week later, the optometrist follows up with the patient and finds that all signs and symptoms of dry eye have been eliminated.

### Example 3: Retrobulbar Injection for Treatment of Macular Edema

A 65-year-old male complains of blurry and distorted vision. The 65-year-old male sees an ophthalmologist who diagnoses them with macular edema. To treat this affliction, the ophthalmologist recommends a once-weekly injection of an injectable composition of the present invention comprising molecular hydrogen (H₂).

The 65-year-old male arrives weekly to the ophthalmologist's office, where he receives a retrobulbar injection of molecular hydrogen. After four consecutive treatments, the patient's signs and symptoms of macular edema have been reduced by 65%. During an examination after the fourth weekly retrobulbar injection, the ophthalmologist finds that the volume of liquid residing under the 65-year-old patient's macula has been reduced by at least 70%.

### Example 4: Gel Treatment for Vitreous Floaters

A 23-year-old female complains to her optometrist at least seeing spots in her field of vision. After an examination, the optometrist determines that the patient is experiencing vitreous floaters. Following this diagnosis, the optometrist implements a gel treatment that includes molecular hydrogen. The gel formulation is applied to the patient's naked eye via a moist swab applicator. The optometrist sends the patient home with 14 doses of molecular hydrogen gel and 14 swab applicators for personal use. She is instructed to apply the molecular hydrogen gel directly to her eyes twice daily.

After a week of twice-daily treatment with the gel-based molecular hydrogen, the patient reports in a follow-up appointment that her vitreous floaters have disappeared. The optometrist examines the patient's eyes and discovers that the build-up of collagen fibers has been reduced by 85%.

### Example 5: Gaseous Treatment for Diabetic Retinopathy

A 55-year-old male who is a type 1 diabetic visits an ophthalmologist complaining of blurred vision. A retinal exam reveals that the patient has diabetic retinopathy. The patient also has an aversion to contact lenses (and other objects placed into contact with the eye). Further, he is afraid of injections. Following diagnosis, the ophthalmologist decides to implement a therapy comprising a gaseous composition of molecular hydrogen.

The ophthalmologist applies the molecular hydrogen gas to the patient through goggles that form an airtight seal over his orbital bones. The patient receives this treatment once weekly, each session consisting of 30 minutes of therapy in which his eyes are enveloped in gaseous molecular hydrogen provided to the airtight goggles from a conduit connected to a gas source.

After six weekly treatments, the ophthalmologist finds that retinal blood vessel blockage has been reduced by 65% and the patient reports that his symptoms of blurred vision have decreased by more than 50%.

### Example 6: Formulation for Oromucosal Administration

In this example, a 70-year-old male visits a primary care provider complaining of oral lesions. Because the patient has a history of tobacco use, the provider suspects that the lesions are potentially malignant. Further examination reveals inflammation and moderate periodontitis.

The provider gives the patient a solution of molecular hydrogen for daily use (i.e., twice per day). The product is provided as an aqueous solution that flows into the oral cavity and covers the oral mucosal with a thin film for gradual absorption of the active agent (H₂). Two weeks later, the provider follows up with the patient and finds that all inflammation is reduced substantially. Lesions have healed but the patient will be monitored regularly for signs of oral cancer.

### Example 7: Formulation for Treating Skin Conditions

In this example, a 45-year-old female visits a primary care provider and presents abnormal skin growth (i.e., moles) on her upper back. The provider suspects precancerous skin and is concerned that the moles will progress into squamous or basal cell carcinoma. The mole is surgically removed. The provider gives the patient a topical solution of molecular hydrogen for daily use (i.e., once per day). Two months later, the provider follows up with the patient and observes the area where the mole was removed. The skin has healed but the patient will be monitored regularly for signs of irregular growth/cancer.

### Example 8: Kit Containing Formulation for Treating Eye Conditions

Embodiments also include a kit that includes one or more of the formulations described herein. In aspects, the ocular delivery system contains a canister design utilizing a saturated or supersaturated Hydrogen storage element. This element provides a therapeutic concentration of Hydrogen in the solution that is then dispensed to the patient as a spray, mist or drop applied topically to a tissue which could benefit from the addition of hydrogen.

The canister can be a glass bottle, plastic bottle, tube design, metal container, can, box design or any numerous shaped vessels. The Hydrogen generating element can be constructed as an individual metal, a combination of metals or alloys that could include a wide range of possible configurations. Possible elemental metals and/or alloys can include Gold, Silver, Beryllium, Magnesium, Calcium, Strontium, Barium, Radium and Aluminum as well as specific metallic substances capable of reacting with water to produce hydrogen in a controlled manner. The elemental metal reacting with water to produce hydrogen can be formed into any number of shapes including as an oval, spherical, cubic, rod, pyramidal, rectangular prism, hollow tube, cross or other geometric shape conducive to the canister design.

In aspects, the element resides inside the canister possibly under pressure and can be located in the center, within a liner, inside the lid, or dispenser opening, within a connected external canister or within an internal sub-chamber. The ocular delivery system canister can be a multi-chambered design, a bagged canister with a liner design or a bag-on-valve system. The composition of the canister can be made of any combination of metals, plastics, rubbers, glass, woods and ceramic materials, foils or a combination therein. The contents of the canister may be stored under pressure or may not and may also contain automatic or manual ingredient mixing devices. The contents of the canister may also be formed to allow electrolysis of water from an external power source. These mixing devices may be internal or external to the canister.

In aspects, elements are arranged in columns of elements with similar properties in the Periodic Table of the elements. The safety of the invention is a major consideration in the selection of the metal generating the hydrogen in a controlled manner. While the alkali metals of the first column including lithium, sodium and potassium are good generators of hydrogen in their reaction with water, other metals from column thirteen of the periodic table are more preferred generators of hydrogen which react less vigorously with water to produce a steady source of hydrogen over time in the container closure. Additionally, members of the second column of alkaline earth metals including magnesium are most preferred for their safe production of hydrogen over time in a therapeutically useful and safe manner.

While the illustrated embodiments are described in the context of a human eye, the present disclosure is not so limited. The delivery system, including the geometry of the container, quick-release seal, and the like may be adapted for the treatment of a variety of animals prone to eye conditions, such as dogs, cats, and other mammals.

The foregoing detailed description will provide those skilled in the art with a convenient road map for implementing various embodiments of the present disclosure. However, it will be appreciated that the particular embodiments described above are only examples, and are not intended to limit the scope, applicability, or configuration of the present disclosure in any way. To the contrary, various changes may be made in the function and arrangement of elements described without departing from the scope of the present disclosure.

As used herein, the term “at least” in reference to a numerical value means ±10% of the given numerical value. Thus, at least 1.0 refers to a range of 0.9-1.1, and at least 10 refers to a range of 9-11, for example.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

Although embodiments of the current disclosure have been described comprehensively in considerable detail to cover the possible aspects, those skilled in the art would recognize that other versions of the disclosure are also possible.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit of this invention.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “at least.” As used herein, the term “at least” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method of treating an individual suffering from a dry eye syndrome comprising administering to the eye of the individual a solution of:
a) molecular hydrogen (H₂); and
b) saline solution or sterile water.

2. The method of claim 1, wherein the composition further comprises oxygen gas (O₂).

3. The method of claim 2, wherein the molecular hydrogen and oxygen gas have a molar ratio (H₂:O₂) of at least 2:1.

4. The method of claim 1, wherein the molecular hydrogen is present in an amount of 0.5 to 10.0 ppm.

5. The method of claim 1, wherein the molecular hydrogen is present in an amount of 5 to 10 ppm.

6. The method of claim 1, wherein the molecular hydrogen is present in an amount of 0.5 to 0.6 ppm.

7. The method of claim 1, further comprising one or more of a mineral oil, an aloe, a polyethylene glycol and a propylene glycol.

8. The method of claim 1, wherein the saline solution is a phosphate buffered saline solution.

9. The method of claim 1, wherein the saline solution is a ringer's solution.

10. The method of claim 1, wherein the pH of the composition is at least 6.5.

11. The method of claim 1, wherein the pH of the composition is at least 7.2-7.4.

12. The method of claim 2, wherein the oxygen gas is at a concentration of at least 9-13 mg/L.

13. The method of claim 2, wherein the oxygen gas is at a concentration of at least 9-13 mg/L and the pH of the composition is at least 7.2-7.4.

14. A method of treating an individual suffering from a dry eye syndrome, wherein an ocular delivery system is provided to the individual, wherein, the ocular delivery system comprises a solution of:
a) molecular hydrogen (H₂);
b) saline solution or sterile water; and,
c) an oxygen gas (O₂);
wherein, the solution is provided in a container having an opening configured to substantially fit over an eye of a subject;
further wherein, the container comprises a quick-release seal removably attached to, and providing a water-proof seal for, the opening of the container; and, the solution is disposed within the container.

15. The method of claim 14, wherein the solution comprises 0.5-0.6 ppm molecular hydrogen (H₂) and saline or sterile water.

16. The method of claim 14, wherein the quick-release seal is deployed to expose the eye of the individual to the solution.

17. The method of claim 14, wherein an effective amount of the solution is administered to an eye of the subject.

18. The method of claim 14, wherein the solution is formulated as a gel, ointment, gas, mist, or spray.

19. A method of treating an individual suffering from a dry eye syndrome, wherein an ocular delivery system is provided to the individual, wherein, the ocular delivery system comprises a solution of:
a) molecular hydrogen (H₂);
b) saline solution or sterile water; and,
goggles configured to be worn airtight over a subject's orbital bones and envelop a subject's eyes,
wherein the goggles are attached to a conduit, and the conduit is attached to a source of gaseous molecular hydrogen; and
wherein the goggles are configured to deliver the solution as a gaseous molecular hydrogen to the subject's eyes from the source of gaseous molecular hydrogen, exposing the subject's eyes to gaseous molecular hydrogen.

20. The method of claim 19, wherein the solution further comprises oxygen gas (O₂).

21. The method of claim 20, wherein the hydrogen gas and oxygen gas have a molar ratio (H₂:O₂) of at least 2:1.

22. The method of claim 20, wherein the molecular hydrogen gas is present in an amount of 0.5 to 10.0 ppm.

23. The method of claim 20, wherein the hydrogen gas is present in an amount of 5 to 10 ppm.

24. The method of claim 1, wherein the composition further comprises one or more of mineral oil, aloe, polyethylene glycol and propylene glycol.

25. The method of claim 1, wherein the composition is administered to one or both eyes of the subject.

26. The method of claim 1, wherein the composition is administered as a drop, an ointment, an eye soak, an emulsion, a gas, a vapor, a mist, an aerosol, a gel, a bottled or canned solution or a bag on valve system.

27. The method of claim 14, wherein the composition is administered with one or more additional medicaments.

28. The method of claim 1, wherein the one or more additional medicaments are selected from antibiotics, steroids, biologics and small molecule therapeutics.
